(19) United States
(12) Patent Application Publication
(10) Pub. No.: US 2012/0225104 A1
(43) Pub. Date: Sep. 6, 2012

(54) COMPOSITION OF ECHIUM OIL AND ESTERS, MANUFACTURING METHOD AND APPLICATION THEREOF

(75) Inventor: Chen-I Pang, Taipei (TW)

(73) Assignee: TAIWAN AC SCIENTIFIC INC., Taipei (TW)

(21) Appl. No.: 13/038,707

(22) Filed: Mar. 2, 2011

(51) Int. Cl. A61K 8/92; A61Q 19/08; A61K 8/04

(52) U.S. Cl. 424/401; 514/560

(57) ABSTRACT

The present invention provides a composition comprising ester carriers and blocks. The ester carriers are composed of Carnauba wax. The carrier has a structure comprising many enclosed regions in a nanometer dimension. The blocks are composed of Echium oil. The blocks are disposed in the enclosed regions. Additionally, the present invention provides applications of such composition, and the applications are related to skin care.

### BACKGROUND OF THE INVENTION

The present invention relates to a composition of Echium oil and esters, the manufacturing method for the composition and the applications of the composition. The present invention particularly relates to a composition that is for purposes of skin care.

Skin care technology is one of the most important technologies for human beings in the modern world. Particularly for female consumers, skin care technology is very much desired, because they have to rely on such technology to prevent their skin from aging so as to show their beauty.

Before the present invention was created, there were some existing technologies made for such need. The U.S. patent application Ser. No. 12/419,397, of which the publication number is 20100256032, provided a composition containing soy as a material for skin care. The U.S. patent application Ser. No. 12/815,958, of which the publication number is 20100254919, on the other hand, provided a turmeric composition as a material for skin care.

The present invention provides an alternative solution to improve skin care technology. Next, the details and embodiments of the present invention will be further explained.

### SUMMARY OF THE INVENTION

The present invention provides a composition having Echium oil and esters, and the composition comprises an ester carrier and many blocks. The ester carrier is made of Carnauba wax. The ester carrier has a structure, and the structure has many nanometer-graded enclosed regions. The blocks are made of Echium oil. The block is disposed in the nanometer-graded enclosed region.

For the above-mentioned composition (first composition), the present invention provides another composition (second composition) that is a kind of application regarding the first composition. The second composition comprises many ester carriers, many blocks and a viscous fluid. The ester carrier is made of Carnauba wax, and it has a structure including many nanometer-graded enclosed regions. The block is made of Echium oil. The block is disposed in the nanometer-graded enclosed region. Additionally, those ester carriers disperse in the viscous fluid.

The present invention also provides a method for manufacturing a composition having Echium oil and esters. The method comprises the following steps. First, provide a first mixture of Echium oil and Carnauba wax. Second, heat the first mixture to a temperature above the melting point of Carnauba wax. Third, add the first mixture into a liquid solution of a surfactant and alcohol. Then, a second mixture will be formed. Fourth, emulsify the second mixture. Finally, let the second mixture cool down.

Next, the details of the embodiments of the present invention will be given.

### BRIEF DESCRIPTION OF THE DRAWINGS

FIG. 1 shows a composition of Echium oil and esters of the present invention.

FIG. 2 shows a composition of Echium oil and esters of the present invention.

FIG. 3 shows a hand of a human being.

### DETAILED DESCRIPTION OF THE INVENTION

### 1. A Composition Having Echium Oil and Esters

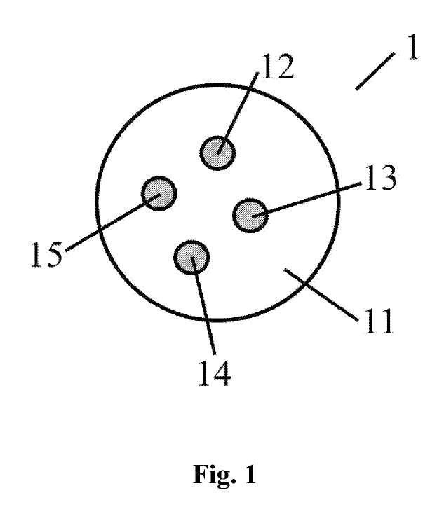

As shown in FIG. 1, the present invention provides a composition 1. The composition 1 is composed of Echium oil and esters. The composition 1 comprises an ester carrier 11 and many blocks 12, 13, 14, 15.

The ester carrier 11 is made of Carnauba wax. The ester carrier 11 has a structure 11. The structure 11 has many nanometer-graded enclosed regions 12, 13, 14, 15.

The blocks 12, 13, 14, 15 are made of Echium oil. That is, the main component of the blocks is Echium oil, and each block may have other additives. The blocks 12, 13, 14, 15 are disposed in the nanometer-graded enclosed regions 12, 13, 14, 15.

The dimension of the ester carrier 11 is in the nanometer level. That means that if the ester carrier 11 is almost spherical, the diameter of the ester carrier 11 is within one micrometer, or almost equal to one micrometer.

The nanometer-graded enclosed regions 12, 13, 14, 15 may be defects among crystal cells of the ester. Thus, the dimension of the enclosed regions is in the nanometer level. That is, if the enclosed region is a three-dimensional object that has a plurality of facets, the longest distance between any two facets is equal to, or smaller than, one micrometer.

The shape of an ester carrier 11 and the shapes of blocks 12, 13, 14, 15 or enclosed regions 12, 13, 14, 15 as shown in FIG. 1 are exemplary. The shape of an ester carrier 11 and the shapes of blocks 12, 13, 14, 15 or enclosed regions 12, 13, 14, 15 may be spherical or of any other kind of shape.

In the present invention, Echium oil is made by the extraction of Echium lycopsis, and was acquired from a U.K.-based company called Croda Chemicals Europe Ltd. Carnauba wax was acquired from a U.S.-based company called Lambent Technologies.

2. A Method for Making a Composition that Includes Echium Oil and Esters

The present invention further provides a method for manufacturing the above-mentioned composition 1. The steps of the method are as follows:
(a) Provide a first mixture that has Echium oil and Carnauba wax.
(b) Heat the first mixture to a temperature above the melting point of Carnauba wax.
(c) Add the first mixture into a liquid solution to form a second mixture, where the liquid solution has a surfactant and an alcohol.
(d) Emulsify the second mixture.
(e) Cool the second mixture down.

In step (a), the present invention uses Echium oil bought from a U.K. company called Croda Chemicals Europe Ltd. Carnauba wax used in the present invention is bought from a U.S. company called Lambent Technologies. The first mixture is made of 20 ml Echium oil and 5 g Carnauba wax.

In step (b), the first mixture is heated to 85 degrees Celsius, so that the first mixture could become a liquid solution. The melting point of Carnauba wax is 85 degrees Celsius. When the temperature of the first mixture reaches 85 degrees Celsius, the first mixture becomes a liquid solution. As a result, the liquid solution has Echium oil and Carnauba wax.

In step (c), the present invention first provides a liquid solution that includes a surfactant and an alcohol. The surfactant used is decyl glucoside, and it is bought from a German company called Cognis. The alcohol used is pentylene glycol, and it can be acquired from a Japan company called Kokyu alcohol Kogyo Co. Ltd. Besides, in the present invention, the liquid solution is made of 9 ml decyl glucoside and 1 ml pentylene glycol. In addition to these two materials just mentioned, the liquid solution further has water.

Then, pour the first mixture into the liquid solution. Now, a second mixture is created. The temperature of the second mixture is kept at 85 degrees Celsius. So, the second mixture is a solution that includes a liquid component and an oil component. Moreover, the liquid component may be a liquid solution of decyl glucoside and pentylene glycol. The oil component may be the first mixture.

In step (d), a pre-emulsification process is first carried out. Put the second mixture into a homogenizer to carry out the pre-emulsification process. While the process is operating, the rotating speed of the blender of the homogenizer is 8000 rpm (rounds per minute). The time for carrying out the process is 150 to 450 seconds.

After the pre-emulsification, the second mixture is poured into a high pressure homogenizer for carrying out a high pressure homogenization (HPH) process. During the HPH process, the pressure used for the high pressure homogenizer is 800 bar. The HPH process takes about 5 to 15 minutes.

Lastly, in step (e), cool down the second mixture to let the temperature of the second mixture reach room temperature. Through the above-mentioned steps, composition 1 having Echium oil and Carnauba wax will be formed.

The particle size of the composition 1 made through the above-mentioned steps ranges from 50 to 100 nanometers. The data of particle sizes may be collected from dynamic light scattering (DLS).

Through the method described above, nine experiments were carried out. The particle size data for those experiments are 81.36, 67.63, 80.65, 93.36, 52.89, 66.26, 68.87, 71.40 and 55.23 nanometers, respectively. The structure of the composition 1 after each experiment is illustrated in FIG. 1.

### 3. Applications of a Composition 1 Having Echium Oil and Esters

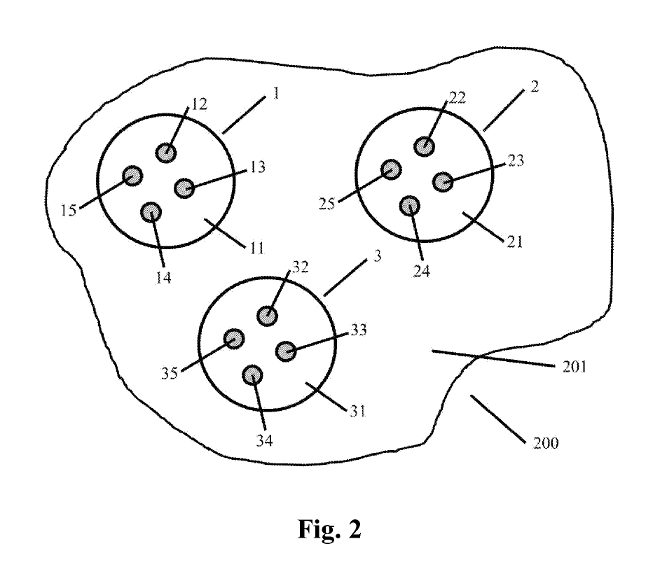

The present invention further provides applications of the composition 1. As shown in FIG. 2, the present invention provides a composition 200 having Echium oil and esters. The composition 200 includes many ester carriers 11, 21, 31, many blocks 12, 13, 14, 15, 22, 23, 24, 25, 32, 33, 34, 35 and a viscous fluid 201.

The ester carriers 11, 21, 31 are made of Carnauba wax. The ester carrier 11, 21, 31 has a structure, and the structure has many nanometer-graded enclosed regions 12, 13, 14, 15, 22, 23, 24, 25, 32, 33, 34, 35. The blocks 12, 13, 14, 15, 22, 23, 24, 25, 32, 33, 34, 35 are made of Echium oil. That is, the main component of the block is Echium oil, and the block may have other additives. The blocks 12, 13, 14, 15, 22, 23, 24, 25, 32, 33, 34, 35 are respectively disposed in the nanometer-graded enclosed regions 12, 13, 14, 15, 22, 23, 24, 25, 32, 33, 34, 35. The viscous fluid 201 may be any kind of cream. The ester carriers 11, 21, 31 may disperse in the viscous fluid 201.

A method for manufacturing the composition 200 is to add the ester carriers 11, 21, 31 into the viscous fluid 201 to mix these materials together.

The composition 200 can be used for keeping skin moist because Echium oil has ingredients good for skin care and those ingredients can penetrate skin. Echium oil has a lot of gamma linoleic acid (GLA), omega-3 long chain fatty acid and stearidonic acid that are necessary fatty acids for skins. Therefore, the composition 200 should provide a positive and healthy influence on skin health.

### 4. Skin Safety Test Regarding the Composition 200

The present invention also provides a report of the skin safety test regarding the composition 200. To carry out the test, first, one sample of the composition 200 is prepared. The sample is a mixture of the composition 1 in three percentage in weight (3 w/w %) and the cream 201 in ninety-seven percentage in weight (97 w/w %). The main components of the cream 201 include Xantham gum and hyaluronic acid. The cream 201 is composed of Xantham gum in twenty percentage in weight (20 w/w %) and hyaluronic acid in ten percentage in weight 10 w/w %). Besides, the cream 201 may further comprise water.

Second, the present invention uses a skin detecting apparatus, Mexameter MX16, supplied by a German company called Courage+Khazaka electronic GmbH., to demonstrate the safety of the sample.

The conditions of the test are as follows:

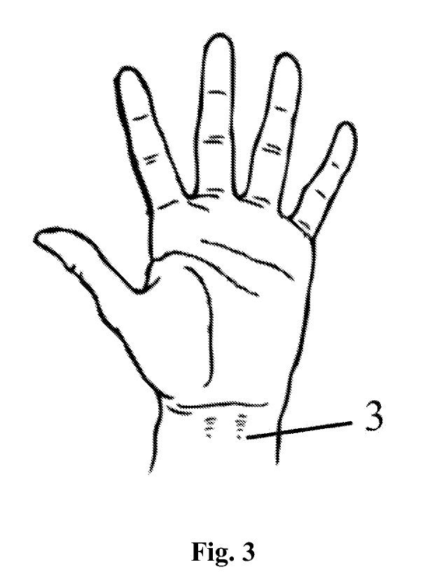

(1) Test Target: wrist 3 (as shown in FIG. 3).

(2) Test Frequency:
1st Time: the measurement is carried out right before the sample was spread over the wrist 3.
2nd Time: the measurement is carried out 24 hours after the sample was spread over the wrist 3.
3rd Time: the measurement is carried out 48 hours after the sample was spread over the wrist 3.

(3) Test Groups:
Group I: the wrist 3 is not coated.
Group II: the wrist 3 is coated by the sample.
Group III: the wrist 3 is coated purely by the composition 1.

Table 1 shows the test results, where the E values (Erythema values) of the skin safety test are reported. The E value represents the index of skin dusts. The E values measured demonstrate that the composition provided by the present invention will not cause negative effects on skins.

TABLE 1

| E value | 1st Time | 2nd Time | 3rd Time |
|---|---|---|---|
| Group I | 32 | 33 | 33 |
| Group II | 33 | 34 | 33 |
| Group III | 33 | 35 | 34 |

### 5. Skin Moisture Test Regarding the Composition 200

The present invention also provides the skin moisture test regarding the composition 200. The present invention uses a skin moisture testing apparatus, Corneometer CM 825, supplied by a German company called Courage+Khazaka electronic GmbH.

To carry out the test, first, two kinds of sample of the composition 200 are prepared. The first sample is a mixture of the composition 1 in three percentage in weight (3 w/w %) and the cream 201 in ninety-seven percentage in weight (97 w/w %). The second sample is a mixture of the composition 1 in ten percentage in weight (10 w/w %) and the cream 201 in ninety percentage in weight (90 w/w %).

For those two kinds of sample, the main components of the cream 201 include Xantham gum and hyaluronic acid. The cream 201 is composed of Xantham gum in twenty percentage in weight (20 w/w %) and hyaluronic acid in ten percentage in weight 10 w/w %). Besides, the cream 201 may further comprise water.

The conditions of the test are as follows:

(1) Test Target: wrist 3 (as shown in FIG. 3).

(2) Test Frequency:
1st Time: the measurement is carried out right before the sample was spread over the wrist 3.
2nd Time: the measurement is carried out 5 minutes after the sample was spread over the wrist 3.
3rd Time: the measurement is carried out 30 minutes after the sample was spread over the wrist 3.
4th Time: the measurement is carried out 60 minutes after the sample was spread over the wrist 3.
5th Time: the measurement is carried out 90 minutes after the sample was spread over the wrist 3.
6th Time: the measurement is carried out 120 minutes after the sample was spread over the wrist 3.

(3) Test Groups:
Group I: the wrist 3 is not coated.
Group II: the wrist 3 is coated by the first sample.
Group III: the wrist 3 is coated by the second sample.
Group IV: the wrist 3 is coated purely by the composition 1.

Table 2 shows the test results, where the H values of the skin moisture test are reported. The unit of an H value is Arbitrary Capacitance Units (A.C.U.). The higher an H value is, the better the skin moisture is kept. The data in Table 2 demonstrate that the compositions 200 provided by the present invention improve skin moisture status because the H values of Groups II and III for the 2nd, 3rd and 4th times are higher than those of Group I.

TABLE 2

| | 1st | | 3rd | | | |
|---|---|---|---|---|---|---|
| H value | Time | 2nd Time | Time | 4th Time | 5th Time | 6th Time |
| Group I | 45 | 43 | 41 | 41 | 41 | 41 |
| Group II | 46 | 57 | 58 | 62 | 45 | 45 |
| Group III | 44 | 55 | 54 | 55 | 46 | 45 |
| Group IV | 45 | 46 | 46 | 45 | 45 | 45 |

Therefore, the present invention has provided a composition of Echium oil and esters, and the composition can apply to skin care.

While the invention has been described by way of example and in terms of preferred embodiment, it is to be understood that the invention is not limited thereto. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A composition having Echium oil and esters, comprising:
an ester carrier, wherein the ester carrier is made of Carnauba wax and has a plurality of nanometer-graded enclosed regions; and
a plurality of blocks, wherein the block is made of Echium oil and is disposed in the nanometer-graded enclosed region.

2. A composition having Echium oil and esters, comprising:
a plurality of ester carriers, wherein the ester carrier is made of Carnauba wax and the ester carrier has a structure, wherein the structure has a plurality of nanometer-graded enclosed regions;
a plurality of blocks, wherein the block is made of Echium oil, and wherein the block is disposed in the nanometer-graded enclosed region; and
a viscous fluid, wherein the ester carriers disperse in the viscous fluid.

3. A method for manufacturing a composition having Echium oil and esters, comprising:
providing a first mixture, wherein the first mixture comprises Echium oil and Carnauba wax;
heating the first mixture at a temperature above the melting point of Carnauba wax;
putting the first mixture into a liquid solution to form a second mixture, wherein the liquid solution comprises a surfactant and an alcohol;
emulsifying the second mixture; and
cooling the second mixture to form a composition having Echium oil and Carnauba wax, wherein the composition comprises:
a Carnauba wax carrier, wherein the Carnauba wax carrier has a plurality of nanometer-graded enclosed regions; and
a plurality of blocks, wherein the block is made of Echium oil and the block is disposed in the nanometer-graded enclosed region.

4. The method as claimed in claim 3, wherein the alcohol is pentylene glycol.

5. The method as claimed in claim 3, further comprising after the first mixture was put into the liquid solution, maintaining the second mixture at a temperature above the melting point of Carnauba wax.

6. The method as claimed in claim 3, wherein a step of emulsifying the second mixture further comprises:
carrying out a pre-emulsification process of the second mixture by a first homogenizer; and
carrying out an emulsification process of the second mixture by a high pressure homogenizer.

7. The method as claimed in claim 3, wherein a rotating speed of the first homogenizer is 8000 rpm.

8. The method as claimed in claim 3, wherein a period of operating the first homogenizer is 150 to 450 seconds.

9. The method as claimed in claim 3, wherein the operating pressure of the high pressure homogenizer is 800 bar.

10. The method as claimed in claim 3, wherein a period of operating the high pressure homogenizer is 5 to 15 minutes.
